(19) United States
(12) Patent Application Publication
(10) Pub. No.: US 2019/0125573 A1
(43) Pub. Date: May 2, 2019

(54) ORAL APPLIANCE

(71) Applicants: Young Hyon Park, Incheon-si (KR); Pasamedi Co., Ltd., Incheon-si (KR)

(72) Inventors: Young Hyon PARK, Incheon-si (KR); Hyun Jin Choi, Incheon-si (KR); Jun Young Park, Incheon-si (KR); Jun Won Park, Incheon-si (KR)

(21) Appl. No.: 15/838,446

(22) Filed: Dec. 12, 2017

(30) Foreign Application Priority Data
Oct. 27, 2017 (KR) 10-2017-0141057

(51) Int. Cl. A61F 5/56

(52) U.S. Cl. CPC A61F 5/566

(57) ABSTRACT

The present invention relates to an oral appliance, and more specifically, to an oral appliance that may remain fastened to the teeth for a long time by co-using a fastening portion and an assistant fastening portion on the molars. According to the present invention, the oral appliance co-uses a first fastening portion that provides a fastening by friction against the molars and a second fastening portion that provides a fastening by a fastening protrusion to remain fastened to the user's teeth although the frictional fastening force weakens over time. The second fastening portion may be configured of a circular protrusion, subjecting the oral appliance to a tiny movement even when worn, and increasing convenience.

### CROSS-REFERENCE TO RELATED APPLICATIONS

This patent application claims priority under 35 U.S.C. § 119 to Korean Patent Application No. 10-2017-0141057, filed on Oct. 27, 2017, in the Korean Intellectual Property Office, the disclosure of which is incorporated by reference herein in its entirety.

### TECHNICAL FIELD

The present invention relates to an oral appliance, and more specifically, to an oral appliance that may remain fastened to the teeth for a long time by co-using a fastening portion and an assistant fastening portion on the molars and that may permit movement to provide a more comfortable feeling when worn by configuring the assistant fastening portion as a circular protrusion.

### DISCUSSION OF RELATED ART

When a human being falls asleep, his body muscles relax. In particular, the relaxation of the tongue, uvula, soft palate, or tonsils may pressurize and narrow the airway. In this case, the air inhaled flows fast as passing through the narrowed airway, vibrating the peripheral soft tissues with the result of snoring. Severe snoring may block the airway during sleep, leading to obstructive sleeping apnea (OSA) which pauses breathing. When either apnea, characterized by complete cessation of airflow for at least 10 seconds at the nose or mouth, or hypopnea, occurs five times or more per hour, it is diagnosed of OSA which may cause hypoxemia, an increase in carbon dioxide in blood, stroke, heart rate anomaly, or sudden death. OSA has also been known as deeply related to diabetes. Moreover, snoring and OSA disturb deep sleep, inducing persistent and chronic daytime fatigue that results in poor performance in the workplace.

To treat snoring and OSA, there have been ongoing research efforts-they are largely classified into surgical treatment and non-surgical treatment. Surgical treatment involves expanding the narrowed airway by surgery. This method, however, suffers from high risk, a likelihood to develop a complication, and frequent recurrence. This way also has the shortcomings of surgery that is irrecoverable once done. Non-surgery treatment encompasses chemical methods, e.g., via use of medications and physical methods, such as those using oral appliances, e.g., mandibular advancement devices (MADs) or continuous positive airway pressure (CPAP).

ACPAP is a sort of ventilator that forces air to flow through the airway to expand the obstructed airway. Despite its superior effects, however, ACPAP is subject to various disadvantages, such as high purchase cost, displeasure in putting on the user's face, and inconvenience in carrying. The MAD which is worn in the user's mouth moves the user's lower jaw forward, allowing the tongue, which is attached to the mandibular bone, to be pulled forward so that the peripheral soft tissues sagged are spread to secure the airway. As the user secures the airway by wearing the MAD, air inhaled flows naturally to the airway while preventing the peripheral soft tissues from vibrating. Thus, snoring reduces or vanishes. This smoothly increases the air flow into the airway to the normal amount, enabling treatment of OSA.

Conventional techniques related to oral appliances are disclosed in Korean Patent No. 10-1074536, titled “Mandibular Advancement Device,” Korean Patent No. 10-1154618, titled “Mandible Induced Jaw Function Device for Orthodontics,” and Korean Patent No. 10-1706648, titled “Orthodontic Instrument for Occlusal Position.” Korean Patent No. 10-1074536 discloses a structure able to adjust the position of a lower teeth fastening part using, e.g., a screw. Korean Patent No. 10-1154618 discloses a structure in which a micro actuator is mounted in the device to restrict the back-and-forth movement of a block attached to the lower jaw along a straight line. However, mounting an electrical or mechanical moving device in a human mouth may damage the human body due to a short circuit or the mechanical parts of the device. Korean Patent No. 10-1706648 discloses an orthodontic instrument for occlusal position in which an upper teeth fastening part and a lower teeth fastening part are connected together via twin blocks, e.g., elastic bodies, to separate the upper jaw from the lower jaw. However, the twin-block structure for separating upper jaw and lower jaw require a separate connecting device because an independent upper jaw device and lower jaw device should be connected together, resulting in the oral appliance being bulky. Further, the need for a precise connection causes it difficult to manufacture the device and maintain the connecting device. Further, since the upper jaw and the lower jaw are overall connected via the connecting device, no precise occlusion is obtained when the upper jaw device and the lower jaw device, which are separated from each other, come in contact with each other, causing malocclusion.

Such oral appliance moves the lower jaw forward, allowing the tongue to naturally be pulled forward and thereby riding the airway of pressure on it by the tongue. However, where the user unconsciously opens his mouth during sleep to breathe in oxygen which is deplete, the lower jaw which has been forced to advance may be moved back to the original position, which may get rid of the effect of preventing snoring. After the oral appliance is worn in the user's mouth, the user may have difficulty in speaking, opening mouth, or yawing. Further, the temporomandibular joint disk is overloaded, causing pain in not only temporalis but also sternocleidomastoid, trapezius, muscles of mastication, or other adjacent muscular system, and temporomandibular disorder. Such side effects may arise not just in the twin block structure which separates the upper jaw from the lower jaw but also in the monoblock structure in which the upper and lower teeth are received in a single body.

To address such issues, the inventors have received patents relating to oral appliances fastened to the upper jaw via friction from the teeth while securing flexibility, which are disclosed in Korean Patent No. 10-1301525, titled “Lower Jaw Advancement Snoring Preventing Oral Appliance Intended for Both Upper Jaw and Lower Jaw Which Permits Slight Movement of Temporomandibular Joint” and Korean Patent No. 10-1463021, titled “Oral Appliance.”

In order to fasten an oral appliance using friction between the molars and the oral appliance to secure the mobility of law jaw, the oral appliance needs to be precisely manufactured for fastening to the molars. Further, straightening teeth tends to take effect in a few weeks to several months, and this weakens the fastening between the teeth and the oral appliance. Therefore, the oral appliance requires an alteration to remain fastened to the teeth, and this causes the hassle to visit the dentist.

### PRIOR TECHNICAL DOCUMENTS

### Patent Documents

Korean Patent No. 10-1074536 (issued on Oct. 17, 2011)

Korean Patent No. 10-1154618 (issued on Jun. 8, 2012)

Korean Patent No. 10-1706648 (issued on Feb. 14, 2017)

Korean Patent No. 10-1301525 (issued on Sep. 2, 2013)

Korean Patent No. 10-1463021 (issued on Nov. 18, 2014)

### SUMMARY

The present invention is intended to address the foregoing issues and an object of the present invention is to provide an oral appliance that allows the user's long-term use by further adopting an assistant fastening portion to keep it stably fastened t the teeth although the frictional fastening force between the molars and the oral appliance weakens. In particular, the present invention aims to provide an oral appliance having the assistant fastening portion formed of a circular protruding wire to enable a tiny movement so that it may be shifted even when worn, providing more convenience.

To achieve the above objectives, according to the present invention, an oral appliance may comprise a body having a shape corresponding to dentition, an upper groove formed in an upper portion of the body to allow upper teeth of an upper jaw to be fitted therein, a lower groove formed in a lower portion of the body to allow lower teeth of a lower jaw to be fitted therein and to allow the lower jaw to advance by a predetermined distance, and a teeth guide part extending from the body to face a side surface of the upper teeth or the lower teeth, wherein the lower groove includes a first fastening portion formed to be firmly fastened to at least one of left-hand and right-hand molars of the lower teeth by friction and a second fastening portion formed to have a fastening member fastened to an interdental portion of at least one of the lower teeth.

The body may be formed of a medical material or a medical resin.

The upper groove may be formed to be spaced apart from the upper teeth not to be firmly engaged with the upper teeth.

The lower groove may further include a lower-jaw incisor groove formed to allow the other lower teeth than the left-hand and right-hand molars to rest thereon.

The first fastening portion and the lower-jaw incisor groove may be bordered by a canine.

The lower-jaw incisor groove may be spaced apart from incisors of the lower jaw not to contact the incisors of the lower jaw.

A distance at which the lower-jaw incisor groove may be spaced apart from the incisors of the lower jaw ranges from 0.5 mm to 2.5 mm along a vertical direction.

A distance at which the lower-jaw incisor groove may be spaced apart from the incisors of the lower jaw ranges from 0.3 mm to 0.5 mm along a horizontal direction.

The teeth guide part may have a first portion extending from molars of the upper jaw or lower jaw longer and a second portion extending from incisors of the upper jaw or lower jaw, the first portion being longer than the second portion.

The fastening member may include a wire buried in the body and a pin-shaped dental wire formed of a circular protrusion extending from the wire and attached onto an interdental portion of the lower teeth.

The fastening member may be formed of a metal, ceramic, or plastic.

The second fastening portion may be formed to overlap the first fastening portion or adjacent the first fastening portion.

The body may have an adjusting hole formed in an outside surface of where the fastening member is positioned to adjust the fastening member from an outside.

The oral appliance may further comprise a tongue tip mount formed in a portion of the body to allow a user's tongue to rest thereon.

According to the present invention, an oral appliance may comprise a body having a shape corresponding to dentition, an upper groove formed in an upper portion of the body to allow upper teeth to be fitted therein, a lower groove formed in a lower portion of the body to allow lower teeth of a lower jaw to be fitted therein and to allow the lower jaw to advance by a predetermined distance, and a teeth guide part extending from the body to face a side surface of the upper teeth or the lower teeth, wherein the upper groove includes a first fastening portion formed to be firmly fastened to at least one of left-hand and right-hand molars of the upper teeth by friction and a second fastening portion formed to have a fastening member fastened to an interdental portion of at least one of the upper teeth.

According to the present invention, the oral appliance co-uses the first fastening portion by friction against the molars and the second fastening portion adopting a fastening protrusion, thus able to remain fastened to the user's teeth although the frictional fastening weakens over time. Further, the oral appliance configures the assistant fastening portion as a circular protruding wire that may lead to a tiny movement, thereby rendered moveable even when worn and increasing convenience.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a perspective view illustrating an upper jaw-fixed oral appliance according to an embodiment;

FIG. 2 is a perspective view illustrating a low jaw-fixed oral appliance according to an embodiment;

FIG. 3 is a cross-sectional view illustrating a first fastening part in an upper jaw-fixed oral appliance according to an embodiment;

FIG. 4A is a front view illustrating an example in which an oral appliance is worn in the mouth;

FIG. 4B is a side view illustrating an example in which an oral appliance is worn in the mouth;

FIGS. 5 and 6, respectively, are a cross-sectional view and partially enlarged view illustrating an example in which a tooth LT of the lower jaw LJ is vertically or horizontally spaced apart while wearing an oral appliance according to an embodiment;

FIG. 7 is a view illustrating a cross section and an enlarged portion in an example where a lower tooth LT of the lower jaw LJ is vertically or horizontally spaced apart while wearing an oral appliance according to another embodiment of the present invention;

FIG. 8 is a view illustrating a cross section and an enlarged portion in an example where a lower tooth LT of the lower jaw LJ is vertically or horizontally spaced apart while wearing an oral appliance according to still another embodiment of the present invention;

FIG. 9 is a rear view illustrating a low jaw-fixed oral appliance according to an embodiment;

FIG. 10 is a top view illustrating a low jaw-fixed oral appliance according to an embodiment; and

FIG. 11 is a cross-sectional view illustrating a molar area of a low jaw-fixed oral appliance according to an embodiment.

### DETAILED DESCRIPTION OF EXEMPLARY EMBODIMENTS

The terminologies used herein are only for describing particular embodiments and are not intended to limit the present invention. Singular forms as used herein include plural forms unless stated otherwise. The term “comprise” as used herein is used to embody a particular characteristic, region, integer, step, operation, element, and/or component without excluding presence or addition of other particular characteristics, regions, integers, steps, elements, components, and/or groups.

The terms “upper,” “top,” “lower,” “bottom,” left,” and “right” as used herein refer to relative relations in positions among the elements shown in the drawings and should not be intended to limit the present invention. The terms “first,” “second,” “third,” and “fourth” as used herein are used in the following detailed description to refer to various elements, regions, or sections, but such elements, regions, or sections should not be limited by the terms. The terms are used merely to distinguish one element, region, or section from another. Therefore, the term “first element,” “first region,” or “first section” as used herein should also be termed a “second element,” “second region,” or “second section” without departing form the scope of the present invention.

Unless defined otherwise, the technical or scientific terms as used herein have the same meaning as those commonly appreciated by one of ordinary skill in the art to which the present invention pertains. The terms defined in dictionaries commonly used may be construed to comply with those set forth herein and relevant technical documents and should not be interpreted overly ideally or formally unless defined otherwise.

Hereinafter, preferred embodiments of the present invention are described in detail with reference to the accompanying drawings.

According to the present invention, an oral appliance may be produced as an upper jaw fixed oral appliance which is fastened to the user's upper jaw or a lower jaw fixed oral appliance which is fastened to the user's lower jaw. Where an upper jaw fixed oral appliance and a lower jaw fixed oral appliance are distinct from each other, they are separately described. However, where embodiments of the present invention are applicable to upper jaw fixed oral appliances or lower jaw fixed oral appliances, the upper jaw fixed oral appliances and the lower jaw fixed oral appliances are described with them collectively referred to as oral appliances.

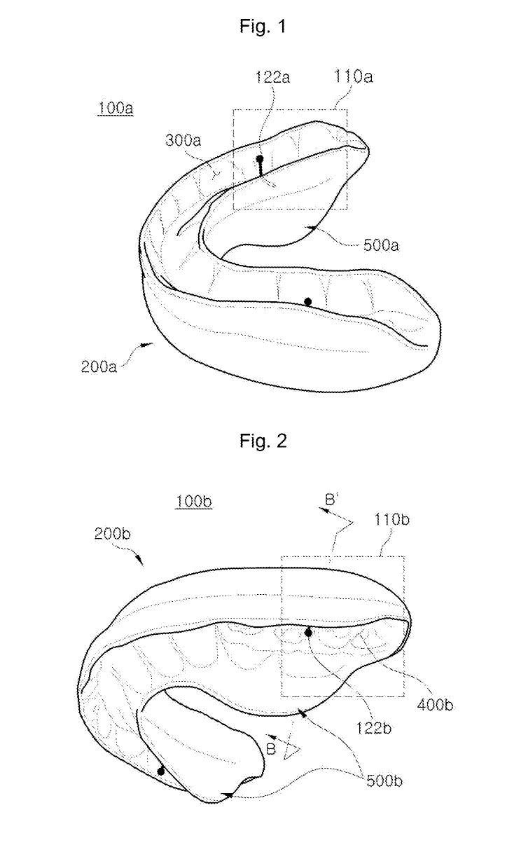

FIG. 1 is a perspective view illustrating an upper jaw-fixed oral appliance according to an embodiment. Referring to FIG. 1, the upper jaw-fixed oral appliance 100a of the present invention is shaped to be fastened to the user's upper jaw UJ and may include a body 200a, an upper groove 300a, and a guide part 500a. The body 200a may have a shape corresponding to the upper dentition UD and lower dentition LD. That is, as shown, the body 200a may be shaped as a horseshoe. Accordingly, the upper dentition UD of the upper jaw UJ representing the upper jaw and the lower dentition LD of the lower jaw LJ representing the lower jaw may correspond to the body 200a.

The upper teeth UT in the upper dentition UD of the upper jaw UJ are fitted into the upper groove 300a formed in the top of the body 200a. Further, although not shown in the drawings, the lower teeth LT in the lower dentition LD of the lower jaw LJ may be fitted into the lower groove formed in the bottom of the body 200a. The lower teeth LT of the lower jaw LJ, although fitted into the lower groove, are not fastened because of no separate fastener. The body 200a may be formed of a dental resin, but without limited thereto, may be formed of silicone or any known material that is not hazardous when worn in the mouth.

The upper jaw-fixed oral appliance 100a including the body 200a according to the present invention may be formed by any known methods, such as by forming a mold for the user's teeth UT and LT and forming shapes corresponding to the mold or by use of a three-dimensional (3D) printer.

The upper groove 300a may be formed in the top of the body 200a. The teeth UT in the upper dentition UD of the upper jaw UJ may be fitted and fastened in the upper groove 300a. To that end, it is preferable that the upper groove 300a is shaped to overall correspond to the upper dentition UD of the upper jaw UJ, i.e., a horseshoe shape, and to correspond to each tooth UT in the upper dentition UD of the upper jaw UJ.

A fastening portion 110a is formed where the molars are positioned in the upper groove 300a to allow at least one molar of the upper teeth UT of the upper jaw UJ to be fastened by friction. The first fastening portion 110a is formed to leave no gap from the user's molar structure of the upper teeth UT of the upper jaw UJ so that the upper teeth UT of the upper jaw UJ tightly fits into the first fastening portion 110a. The upper jaw-fixed oral appliance 100a may be fastened by friction of the molars of the upper teeth UT of the upper jaw UJ to the first fastening portion 110a. Accordingly, unless a larger external force than the friction is applied between the upper groove 300a and the upper teeth UT of the upper jaw UJ, the molars fitted into the first fastening portion 110a would not be escaped from the upper groove 300a of the upper jaw-fixed oral appliance 100a.

The first fastening portion 110a may include a second fastening portion 122a that provides a fastening force equal or smaller than the fastening force of the first fastening portion 110a. The second fastening portion 122a is a portion to provide an additional fastening force to allow the upper jaw-fixed oral appliance 100a to remain fastened to the molars although the first fastening portion 110a is worn or loosens and thus loses the frictional force against the molars. Thus, the fastening force between the second fastening portion 122a and the upper jaw UJ is configured to be preferably smaller than—but may also be the same as—the fastening force of the first fastening portion 110a.

The second fastening portion 122a may be fastened to the molars of the upper jaw UJ in various manners; it would be efficient to use other methods than by friction. For example, where the upper teeth (UT) of the upper jaw UJ are fitted into the upper groove 300a, there may be included a pin-shaped fastening protrusion attachable to the interdental portion between the molars among the upper teeth UT of the upper jaw UJ. The pin-shaped fastening protrusion may be formed of a metallic or heavy-duty ceramic or plastic dental wire.

Where the second fastening portion 122a is formed of a fastening protrusion using a dental wire, its end may be formed of a circular protrusion sized to correspond to the interdental gap. A portion of the wire extending from the circular protrusion may be buried inside the body 200a of the upper jaw-fixed oral appliance 100a to allow the second fastening portion 122a to be stably fixed to the oral appliance 100a.

In this case, an adjusting hole 120a may be formed in the outside surface of the second first fastening portion 122a to allow the wearer of the upper jaw-fixed oral appliance 100a to fit or escape the circular protrusion of the second fastening portion 122a to/from the intermolar portion. This is intended for allowing the wearer to correct the position when the circular protrusion of the second fastening portion 122a is not properly fitted to the intermolar portion. Accordingly, the wearer may modify or adjust the position or attachment of the second fastening portion 122a through the hole 120. However, the adjusting hole 120a is not necessarily required but may rather be omitted given the state of the upper jaw-fixed oral appliance 100a being fastened according to the present invention.

FIG. 2 is a perspective view illustrating a low jaw-fixed oral appliance according to an embodiment. Referring to FIG. 2, the low jaw-fixed oral appliance 100b may be structured symmetrical with the upper jaw-fixed oral appliance 100a, thus including a body 200b, a lower groove 400b, and a guide part 500b to allow it to be fastened to the user's lower jaw LJ.

The body 200b and the guide part 500b may symmetrically have the same structure as those of the upper jaw-fixed oral appliance 100a. The lower groove 400b may be formed in the bottom of the body 200b. The lower teeth LT in the lower dentition LD of the lower jaw (UJ) may be fitted and fastened in the lower groove 400b. To that end, the lower groove 400b may have a shape to overall correspond to the lower dentition (LD) of the lower jaw (LJ)—i.e., shaped as a horseshoe and corresponding in shape to each tooth LT of the lower dentition (LD) of the lower jaw (LJ) as set forth above. Like the upper groove 300a of the upper jaw-fixed oral appliance 100a, the lower groove 400b may be formed in the bottom of the body 200b by various methods, e.g., forming a mold for the user's lower teeth LT followed by forming a shape corresponding to the mold or by use of a 3D printer or any other known methods.

A first fastening portion 110b may be formed in the lower groove 400b of the low jaw-fixed oral appliance 100b to fasten at least molars among the lower teeth (LT) of the lower jaw (LJ) by friction. The first fastening portion 110b formed in the lower groove 400b may be formed to leave no gap from the user's molar structure among the lower teeth (LT) of the lower jaw (U). Accordingly, among the lower teeth (LT) of the lower jaw (LJ), the molars may be fitted into the first fastening portion 110b of the lower groove 400b and may thus be fixed stably by friction. After the first fastening portion 110b is fastened to the molars of the lower jaw (LJ), the molars of the lower jaw (LJ) would not escaped off the lower groove 400b absent otherwise application of a larger external force than the frictional force between the lower groove 400b and the molars of the lower jaw (LJ).

The first fastening portion 110b may include a second fastening portion 122b having a fastening force smaller or equal to the first fastening portion 110b. The second fastening portion 122b formed in the lower groove 400b provides an additional fastening force so that the low jaw-fixed oral appliance 100b remains fastened to the molars of the lower jaw (U) although the first fastening portion 110b is worn or loosens and thus loses the frictional force against the molars. Accordingly, the fastening force between the second fastening portion 122b and the molars of the lower jaw (LJ) may be configured to be smaller than, or equal to, the fastening force of the first fastening portion 110b. The second fastening portion 122b may be formed of a pin-shaped fastening protrusion attachable to an interdental portion that meets the gum between the molars of the lower jaw (LJ). The pin-shaped fastening protrusion may be formed of a metallic, or heavy-duty ceramic or plastic dental wire.

In the low jaw-fixed oral appliance 100b, the molars of the lower jaw (LJ) may be fitted and fastened into the first fastening portion 110b of the lower groove 400b. In contrast, the upper groove (not shown in the figure) may be configured to allow the upper teeth (UT) of the upper jaw (UJ) to be fitted, but not fixed, thereto.

An adjusting hole (not shown) may be formed, or not formed, in the outside surface of the second fastening portion 122b to allow the wearer of the low jaw-fixed oral appliance 100b to fit or escape the circular protrusion of the second fastening portion 122b to/from the intermolar portion. Thus, the wearer may correct or adjust the position or attachment of the second fastening portion 122b through the adjusting hole. Likewise, the adjusting hole is not necessarily required but may rather be omitted considering the state of the low jaw-fixed oral appliance 100b being fastened according to the present invention.

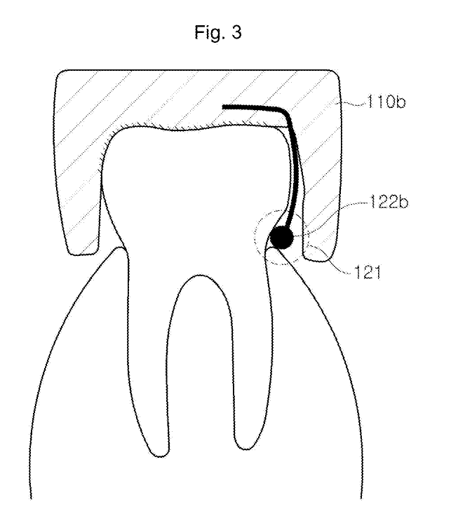

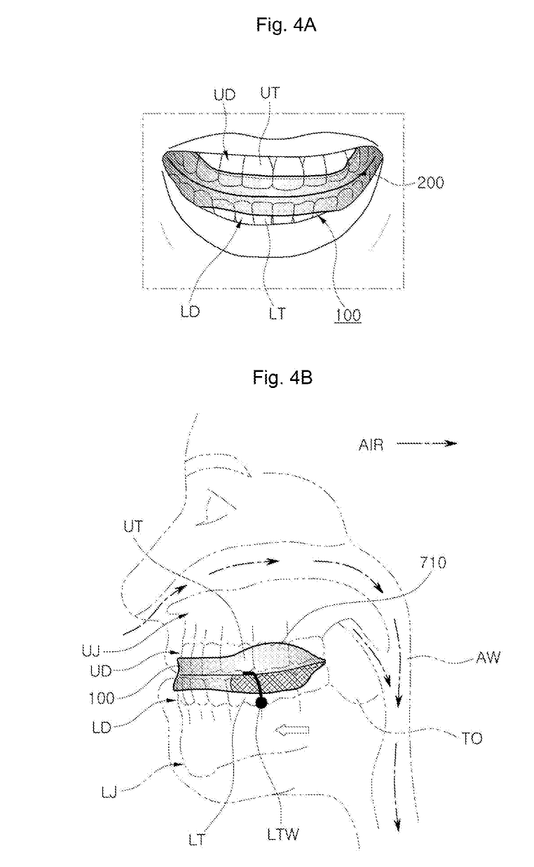

FIG. 3 is a cross-sectional view (taken along line B-B′ of FIG. 2) illustrating a first fastening portion 110b in a low jaw-fixed oral appliance according to an embodiment. Referring to FIG. 3, the first fastening portion 110b formed in the lower groove 400b may be fastened by friction to at least one molar among the lower teeth (LT) of the lower jaw (LJ). In this case, the first fastening portion 110b may be fastened by friction to the crown of the molars in the lower grove 400b. Further, the first fastening portion 110b may include the second fastening portion 122b. The second fastening portion 122b may be formed of a fastening protrusion engageable to the interdental portion 121 of the molars of the lower jaw (LJ).

The fastening protrusion may include a wire portion buried and fastened in the body 200b and a circular protrusion fastened to the interdental portion 121 between the molar of the lower jaw (LJ) and the gum. While the low jaw-fixed oral appliance 100b has the fastening protrusion in the lower groove 400b, the upper jaw-fixed oral appliance 100a has the fastening protrusion 300a in the upper groove 300a. Where the second fastening portion 122b is formed of a pin-shaped fastening protrusion, and the circular protrusion engages to the interdental portion 121 of the molar of the lower jaw (LJ), the fastening of the first fastening portion 110b by friction may be reinforced. Accordingly, although the frictional force of the first fastening portion 110b weakens, the oral appliance 100b of the present invention may stably remain fastened to the wearer's teeth.

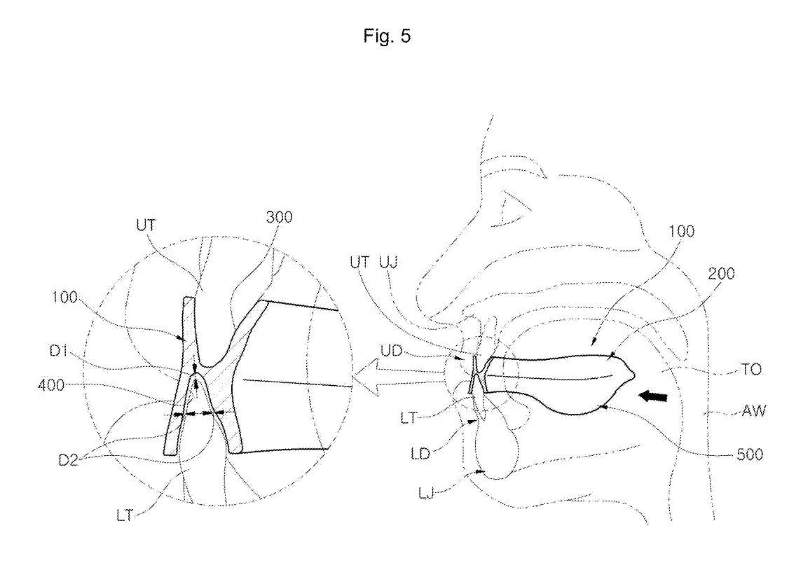

FIG. 4A and FIG. 4B, respectively, are a front view and side view illustrating an example in which an oral appliance is worn in the mouth according to an embodiment. Here, the oral appliance may be an upper jaw-fixed oral appliance 100a or a low jaw-fixed oral appliance 100b (they are collectively referred to as “oral appliance” and denoted by reference number 100). It would be apparent that the description, although focusing only on one of the two types, may be applicable to the other type with the position changed. Referring to FIG. 4, in the low jaw-fixed oral appliance 100b, if the lower teeth (LT) of the lower jaw (LJ) are fitted into the lower groove 400b, the lower jaw (LJ) may be shifted forward by a predetermined distance, resultantly positioning the incisors of the lower jaw LJ a predetermined distance ahead of the incisors of the upper jaw (UJ). In turn, the tongue TO attached to the lower jaw (L) is also shifted forwards along with the lower jaw (LJ), thereby taking a position not to obstruct the airway (AW). In the tongue's position without obstructing the airway (AW), a smooth airflow may be achieved through the airway (AW), and snoring and sleep apnea may be prevented which might otherwise arise. FIG. 4 illustrates an example in which the molars of the lower jaw (U) are fastened by friction in the lower groove 400b of the low jaw-fixed oral appliance 100b.

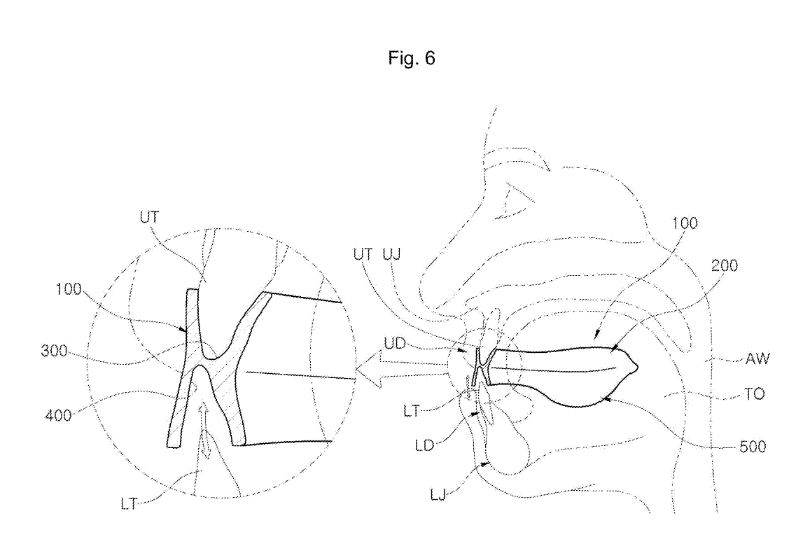

FIGS. 5 and 6, respectively, are a cross-sectional view and partially enlarged cross-sectional view illustrating an example in which the lower teeth (LT) of the lower jaw (LJ) are vertically or horizontally spaced apart while an oral appliance is worn according to an embodiment. Upon wearing the upper jaw-fixed oral appliance 100a, at least one molar of the upper teeth (UT) of the upper jaw (UJ) is fastened to the first fastening portion 110a of the upper groove 300a, the lower groove is vertically or horizontally spaced apart from the lower teeth (LT) at a predetermined interval D1 or D2, and the guide part may extend downwards from the body. Conversely, upon wearing the low jaw-fixed oral appliance 100b, at least one molar of the lower teeth (LT) of the lower jaw (U) is fastened to the first fastening portion 110b of the lower groove 400b, the upper groove is vertically or horizontally spaced apart from the upper teeth (UT) of the upper jaw (UJ) at a predetermined interval D1 or D2, and the guide part may extend upward of the upper jaw (UJ) from the body.

The oral appliance 100 corresponding to one of the upper jaw-fixed oral appliance 100a or the low jaw-fixed oral appliance 100b is configured so that some teeth LT oriented towards the incisors are vertically or horizontally spaced apart at a predetermined interval D1 or D2 from the lower groove 400 to secure the mobility of the lower jaw (LJ) although the lower teeth (LT) of the lower jaw (LJ) are fitted and fastened in the lower groove 400.

As such, leaving the interval D1 or D2 in the vertical or horizontal direction between the lower groove 400 and the lower teeth (LT) of the lower jaw (LJ) allows the lower teeth (LT) of the lower jaw (LJ) to escape from the lower groove and move downwards according to the user's movement, resultantly allowing the lower jaw (LJ) to move downwards. Since the lower jaw (LJ) may naturally be moved downwards although the lower jaw (LJ) is in the position of having advanced, the user wearing the oral appliance 100 of the present invention may be released from stiffness of ambient muscles and ligaments.

Meanwhile, as contacting the teeth of the lower jaw (LJ), the guide part 500 is subject to natural vertical movement by which it stops descending and moves upwards. Such reciprocal motion of the lower jaw (LJ) may repeat while the user is sleeping in the oral appliance 100 of the present invention, contributing to the release of stiffness of related muscles and ligaments. Accordingly, the oral appliance 100 of the present invention may prevent pain, malocclusion, and temporomandibular disorder without the stiffness of juxtaoral muscles and ligaments despite the user's long-term wearing while leading to a better airflow through the airway and hence treating snoring and sleep apnea.

The interval D1 at which the lower groove 400 is vertically spaced apart from the lower teeth (LT) of the lower jaw (LJ) may range from 0.5 mm to 2.5 mm. A research published in relation with the interval that naturally occurs between the upper teeth (UT) of the upper jaw (UJ) and lower teeth (LT) of the lower jaw (LJ) during sleep reveals that the interval may range from about 2 mm to about 4 mm. Meanwhile, the interval between the upper groove 300 and the lower groove 400 may be about 1.5 mm. Accordingly, if the interval D1 at which the lower groove 400 is vertically spaced apart from the lower teeth (LT) of the lower jaw (LJ) is less than 0.5 mm, the interval between the upper teeth (UT) of the upper jaw (UJ) and the lower teeth (LT) of the lower jaw (LJ) may become smaller than 2 mm which is a natural one. In this case, the brain may determine that the body 200 fitted between the upper teeth (UT) of the upper jaw (UJ) and the lower teeth (LT) of the lower jaw (LJ) is food and may thus trigger a masticatory movement that cuts and grinds food. Hence, the interval D1 at which the lower groove 400 is vertically spaced apart from the lower teeth (LT) of the lower jaw (LJ) preferably ranges from 0.5 mm to 2.5 mm.

Meanwhile, the interval D2 at which the lower groove 400 is horizontally spaced apart from the lower teeth (LT) of the lower jaw (LJ) may range from 0.3 mm to 0.5 mm. Since the lower groove is 0.3 mm to 0.5 mm spaced apart from the lower teeth (LT) of the lower jaw (LJ) at one side thereof along the horizontal direction, it may be 0.6 mm to 1.0 mm spaced apart at both sides thereof. If the interval D2 at which the lower groove 400 is horizontally spaced apart from the lower teeth (LT) of the lower jaw (LJ) is less than 0.3 mm, the lower teeth (LT) of the lower jaw (LJ) may be fastened in the lower groove 400 by friction, and the lower teeth (LT) of the lower jaw (LJ) might not be escaped from the lower groove 400 and moved downwards when it need.

If the interval D2 at which the lower groove 400 is horizontally spaced apart from the lower teeth (LT) of the lower jaw (U) exceeds 0.5 mm, the lower teeth (LT) of the lower jaw (J) may freely be shifted in the lower groove but cannot fully escape from the lower groove 400. Accordingly, the interval D2 at which the lower groove 400 is horizontally spaced apart from the lower teeth (LT) of the lower jaw (LJ) preferably ranges from 0.3 mm to 0.5 mm.

The range of the interval D1 or D2 at which the lower groove 400 is vertically or horizontally spaced apart from the lower teeth (LT) of the lower jaw (LJ) may be varied from the above values depending on the angle of slope of the overall occlusion surface, degree of protrusion of teeth, the state of teeth being inclined inwards or outwards, or the position of teeth escaping off the overall dentition for each user. Further, the lower groove 400 may be spaced apart from the lower teeth (LT) of the lower jaw (LJ) in the front-back horizontal direction or in the left-right horizontal direction.

Meanwhile, the guide part 500 may extend downwards from the body 200. For example, the guide part 500 may extend 3 mm to 5 mm down from the body 200. Yet, the guide part 500 is not particularly limited as extending in a certain length, but rather in any length depending on the user's oral condition.

As set forth above, wearing the upper jaw-fixed oral appliance 100a or low jaw-fixed oral appliance 100b of the present invention, the lower teeth (LT) of the lower jaw (LJ) may be naturally moved downwards from the lower groove 400 while being in the position of having advanced in a predetermined distance. The downward movement of the lower jaw (LJ) may stop as the guide part 500 comes in contact with the teeth of the lower jaw (LJ), and the lower jaw (LJ) may move up and fit into the lower groove 400—as such, natural up-and-down motion can be attained. In this case, the guide part 500 may guide the lower teeth (LT) of the lower jaw (LJ) to easily and quickly fit into the lower groove 400b 400. To that end, the guide part 500 may extend to partially cover the gum underneath the molars among the lower teeth (LT) of the lower jaw (LJ) inside the body 200. The guide part 500 is not limited to a particular shape and may rather be formed in any other shapes that may achieve the above-described functions.

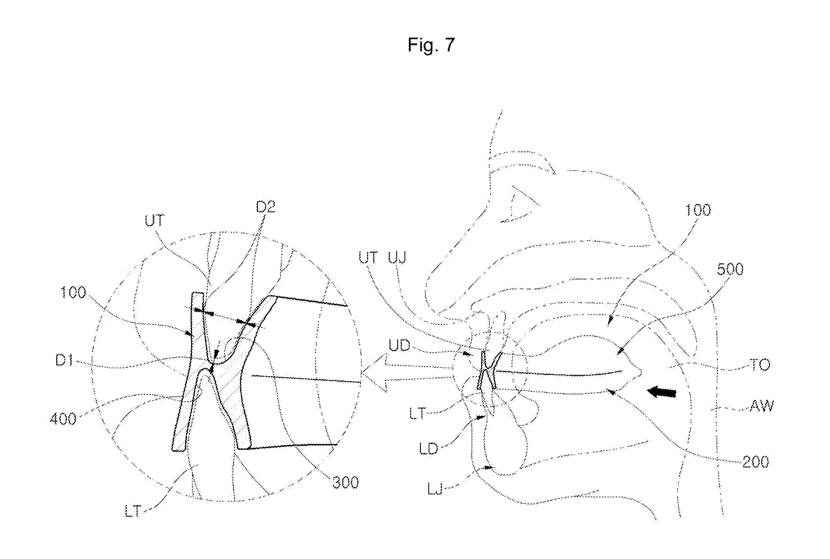

FIG. 7 illustrates a cross-sectional view and partially enlarged cross-sectional view illustrating an example in which lower teeth (LT) of the lower jaw (U) are vertically or horizontally spaced apart while an oral appliance 100 is worn in the mouth according to still another embodiment of the present invention. Although in the embodiment of FIG. 7 an the low jaw-fixed oral appliance 100b is described in which the lower teeth (LT) of the lower jaw (LJ) are fitted and fastened in the lower groove 400, the upper groove 300 is vertically or horizontally spaced apart from the upper teeth (UT) of the upper jaw (UJ) at a predetermined interval D1 or D2, and the guide part 500 extends upwards from the body 200, the same effects may be achieved for the upper jaw-fixed oral appliance 100a. Accordingly, the description is centered on the distinct features, with the description made above in connection with FIGS. 5 and 6 applied to the other features.

In the oral appliance 100 of the present invention, among the lower teeth (LT) of the lower jaw (LJ), molars may be fastened by friction to the first fastening portion 110 of the lower groove 400. Further, the fastening protrusion formed in the first fastening portion 110b, as the second fastening portion 122b, may provide an additional fastening force. The lower groove 400 may be shaped to correspond to the lower teeth (LT) of the lower jaw (LJ) while leaving no gap in some molars of the lower teeth (LT) of the lower jaw (U).

Accordingly, among the lower teeth (LT) of the lower jaw (U), the molars may be tightly fitted into the lower groove 400 by the first fastening portion 110b, and although the first fastening portion 110b is worn or loosened, an additional fastening force is provided by the second fastening portion 122b. In turn, absent application of an external force stronger than the fastening force produced by the fastening protrusion of the second fastening portion 122b and the frictional force between the first fastening portion 110b and the molars of the lower jaw (U), the lower teeth (LT) of the lower jaw (LJ) would not be escaped off the lower groove 400.

Meanwhile, the upper groove 300 may be vertically or horizontally spaced apart from the upper teeth (UT) of the upper jaw (UJ) at a predetermined interval D1 or D2, and the guide part 500 may extend upwards from the body 200. In the low jaw-fixed oral appliance 100b, the upper teeth (UT) of the upper jaw (UJ) may be fitted into the upper groove 300 but not fastened to freely ascend or descend. By such configuration, the upper teeth (UT) of the upper jaw (UJ) may relatively move in the upper groove 300, thus allowing the lower jaw (U) along with the oral appliance 100 of the present invention to naturally move downwards.

If the guide part 500 reaches some molars of the upper teeth (UT) of the upper jaw (UJ) escaped off the upper groove 300 while the lower jaw (U) moves down along with the oral appliance 100, the lower jaw (LJ) stops moving. Thereafter, the upper jaw (UJ) fits into the upper groove 300 where it is positioned before escaping off, and the lower jaw (U) may natural ascend, enabling other stiff muscles and ligaments to relax. At this time, the guide part 500 may guide the upper teeth (UT) of the upper jaw (UJ) to easily and quickly fit into the upper groove 300.

Such reciprocal motion of the lower jaw (LJ) may repeat while the user is sleeping in the oral appliance 100 of the present invention, thus releasing the stiffness of muscles and ligaments that has occurred as the lower jaw (U) advances.

In this case, meanwhile, the interval D1 at which the upper groove 300 is vertically spaced apart from the upper teeth (UT) of the upper jaw (UJ) may range from 0.5 mm to 2.5 mm. The interval D2 at which the upper groove 300 is horizontally spaced apart from the upper teeth (UT) of the upper jaw (UJ) may range from 0.3 mm to 0.5 mm.

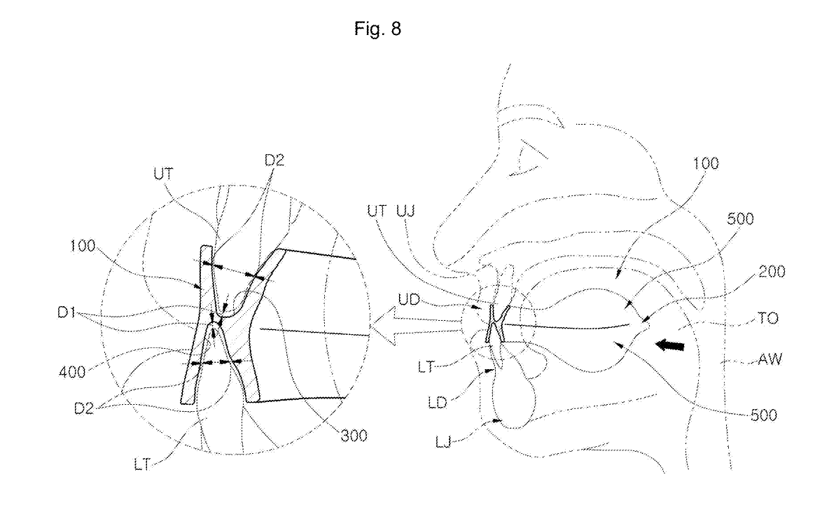

FIG. 8 illustrates a cross-sectional view and partially enlarged cross-sectional view illustrating an example in which the lower teeth (LT) of the lower jaw (LJ) are vertically or horizontally spaced apart while an oral appliance 100 is worn in the mouth according to still another embodiment of the present invention. The embodiment of FIG. 8 differs in that the upper groove 300 is vertically or horizontally spaced apart from the upper teeth (UT) of the upper jaw (UJ) at a predetermined interval D1 or D2, the lower groove 400 is vertically or horizontally spaced apart from the lower teeth (LT) of the lower jaw (LJ) at a predetermined interval D1 or D2, and the guide part 500 extends up and down from the body 200 in a predetermined distance. Accordingly, the description focuses primarily on distinct features, with the description made above in connection with FIGS. 5 to 7 applied to the other features.

The oral appliance 100 as shown in FIG. 8 may be used in the cases where the upper dentition (UD) of the upper jaw (UJ) or lower dentition (LD) of the lower jaw (U) or their teeth UT and LT are in a poor condition, and when fastened t the upper groove 300 or lower groove 400 by friction, a tooth loss is likely or where it is hard to secure a sufficient amount of frictional force by the first fastening portion 110 due to a short length of crown in the upper jaw or lower jaw. In this case, such a configuration may be made that the second fastening portion 122 provides a sufficient amount of fastening force to back up the weak frictional force of the first fastening portion 110.

In this case, any one of the upper teeth (UT) of the upper jaw (UJ) or the lower teeth (LT) of the lower jaw (LJ) may be brought in more tight contact with the upper groove 300 or lower groove 400, and the side relatively less tightly contacting may be moved in the upper groove 300 or lower groove 400. Accordingly, the lower jaw (LJ) may naturally be moved to release the stiffness of muscles and ligaments.

For example, where among the upper teeth (UT) of the upper jaw (UJ), molars are fastened relatively strong in the upper groove 300 by the first fastening portion 110a and the second fastening portion 122a, the lower jaw (U) may be reciprocally moved by the method as described above in connection with FIGS. 5 and 6. Conversely, where the molars among the lower teeth (LT) of the lower jaw (LJ) are fastened relatively strong in the lower groove 400 by the first fastening portion 110b and the second fastening portion 122b by the low jaw-fixed oral appliance 100b, the lower jaw (LJ) may do reciprocal motion in the manner described above in connection with FIG. 7. Such reciprocal motion of the lower jaw (LJ) may repeat while the user is sleeping in the oral appliance 100 of the present invention.

This may relieve the stiffness of relevant muscles and ligaments as the lower jaw (LJ) advances or moves, and wearing the oral appliance 100 would not cause pain or malocclusion, or temporomandibular disorder. Further, a smooth air flow may occur through the airway (AW), mitigating symptoms, such as snoring or sleep apnea.

In this case, also, the interval D1 at which the upper groove 300 is vertically spaced apart form the upper teeth (UT) of the upper jaw (UJ) and the interval D1 at which the lower groove 400 is vertically spaced apart from the lower teeth (LT) of the lower jaw (LJ) may range from 0.5 mm to 2.5 mm. The interval D2 at which the upper groove 300 is horizontally spaced apart from the upper teeth (UT) of the upper jaw (UJ) and the interval D2 at which the lower groove 400 is spaced apart from the lower teeth (LT) of the lower jaw (LJ) may range from 0.3 mm to 0.5 mm.

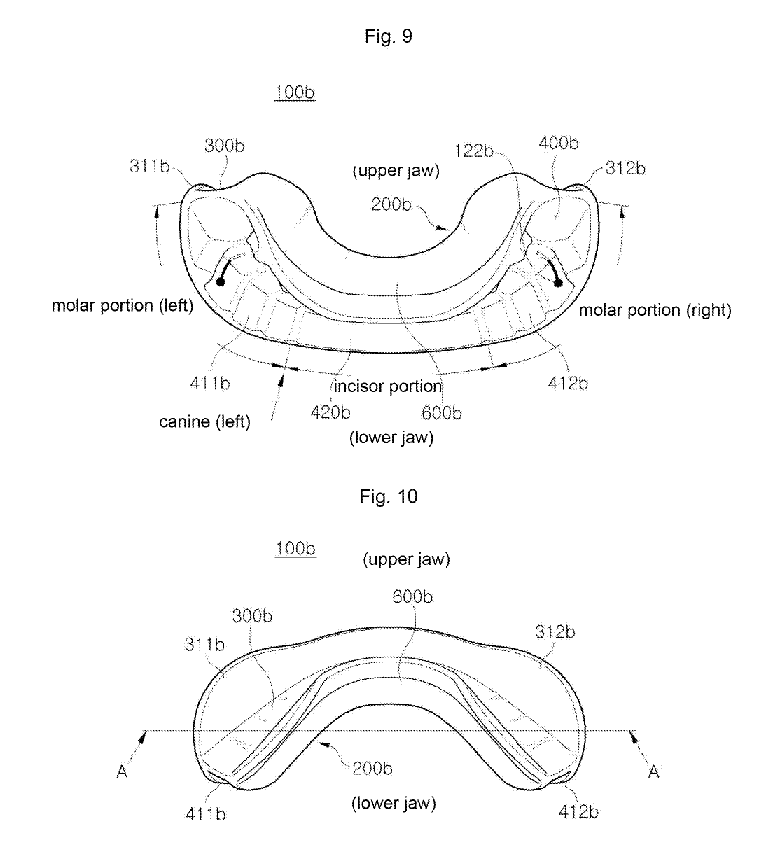

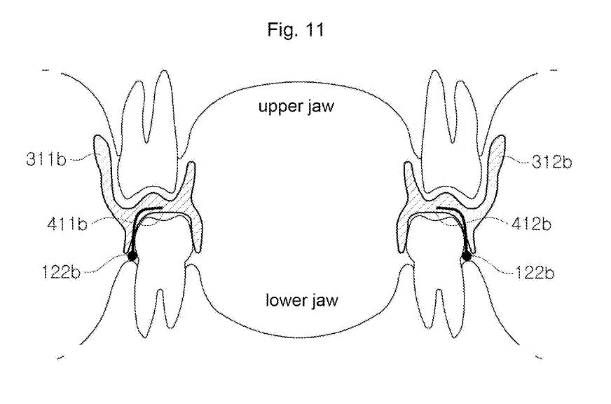

FIGS. 9 and 10, respectively, are a rear view and top view illustrating a low jaw-fixed oral appliance according to an embodiment. Referring to FIGS. 9 and 10, the low jaw-fixed oral appliance 100b may include a body 200b formed of a medical resin or other harmless materials. The body 200b may include an upper groove 300a, teeth guide parts 311b and 312b, a lower groove 400b, and a tongue tip mount 600b. The lower groove 400b may include lower jaw molar fastening grooves 411b and 412b where among the lower teeth (LT) of the lower jaw (LJ) molars are fastened by friction and a lower jaw incisor groove 420b where among the lower teeth (LT) of the lower jaw (LJ) incisors rest. The lower jaw molar fastening grooves 411b and 412b are portions included in the above-described first fastening portion 110b.

Among the left- and right-hand molars of the lower jaw (LJ), at least one molar is firmly engaged into the lower jaw molar fastening grooves 411b and 412b. In contrast, the lower jaw incisor groove 420b is preferably configured to be engaged with the incisors of the lower jaw (J) but not inside the lower jaw incisor groove 420b. The left-hand and right-hand canines may be positioned at the borders between the lower jaw incisor groove 420b and the lower jaw molar fastening grooves 411b and 412b.

Meanwhile, the pin-shaped fastening protrusion constituting the second fastening portion 122b may be provided in the first fastening portion 110b or be positioned between the lower jaw incisor groove 420b and each lower jaw molar fastening groove 411b and 412b. Accordingly, the low jaw-fixed oral appliance 100b of the present invention may simultaneously provide a frictional force against the molars of the lower jaw (LJ) and an assistant fastening force using the second fastening portion 122b.

Meanwhile, the upper groove 300a and the lower groove 400b, respectively, may allow the upper teeth (UT) of the upper jaw (UJ) and the lower teeth (LT) of the lower jaw (LJ) to rest thereon. In this case, the lower groove 400b is formed to be positioned ahead of the user's normal occlusal surface so that the lower teeth (LT) of the lower jaw (LJ) of the user wearing the low jaw-fixed oral appliance 100b advances further before wearing the low jaw-fixed oral appliance 100b. Accordingly, wearing the low jaw-fixed oral appliance 100b, the lower teeth (LT) of the lower jaw (LJ) of the user moves forwards, resultantly expanding the user's airway (AW) and hence enabling a smooth airflow while breathing during sleep. This helps to prevent or mitigate snoring during sleep.

The teeth guide parts 311b and 312b are formed to allow the upper dentition (UD) of the upper jaw (UJ) or lower dentition (LD) of the lower jaw (LJ) to, although shifted, be positioned in place in the upper groove 300b or lower groove 400b of the body 200b. In the low jaw-fixed oral appliance 100b according to the present invention, the upper dentition (UD) of the upper jaw (UJ) is not firmly engaged into the upper groove 300b nor fastened inside the upper groove 300b. Thus, the upper dentition (UD) of the upper jaw (UJ) may escape off the upper groove 300b, able to freely move within a predetermined range. Therefore, the user's long-term wearing of the low jaw-fixed oral appliance 100b would not stiffen the juxtaoral muscles or ligaments, preventing pain, malocclusion, or temporomandibular disorder that may otherwise occur.

Meanwhile, a need exists for allowing the upper dentition (UD) of the upper jaw (UJ) or lower dentition (LD) of the lower jaw (LJ) to be positioned in place inside the upper groove 300b or lower groove 400b. Thus, it is preferable that the teeth guide parts 311b and 312b are formed to extend from the body 200b where the upper teeth (UT) of the upper jaw (UJ) or the lower teeth (LT) of the lower jaw (IJ) contact the side surfaces. In this case, the teeth guide parts 311b and 312b may be formed to be longer at the molars of the upper jaw (UJ) or lower jaw (U) than at the incisors. Further, the tongue tip mount 600b may be shaped as a cavity to allow the user's tongue tip to stably rest frontward of the mouth in the low jaw-fixed oral appliance 100b.

Various conventional oral appliances, e.g., those for preventing snoring, lacking a space for placing the user's tongue in a stable position. This may cause the user's tongue to originally shift in a rear position or to be less adapted to the oral appliance. To address such issues, the oral appliance 100b of the present invention provides the tongue tip mount 600b to allow the user's tongue tip to rest forwards in a stable position.

FIG. 11 is a cross-sectional view illustrating a molar area of a low jaw-fixed oral appliance according to an embodiment. The cross-sectional view of FIG. 11 is about a tangential one to the user's front face among planes including the straight line connecting the user's left-hand molar (A′ position) and right-hand molar (A position). Referring to FIG. 11, the left-hand molar of the lower jaw (LJ) is mounted on the inside surface of the left-hand lower jaw molar fastening groove 411b, and the right-hand molar of the lower jaw (LJ) is mounted on the inside surface of the right-hand lower jaw molar fastening groove 412b. Further, as at least one of the molars of the lower jaw (LJ) is firmly engaged to the inside surfaces of the lower jaw molar fastening grooves 411b and 412b by friction, the oral appliance 100b may be prevented from escaping off too easily. Further, additional use of the pin-shaped fastening protrusion, as the second fastening portion 1200b, together with the lower jaw molar fastening groove 411b or 412b, i.e., the first fastening portion 110b, enables a more secure fastening between the lower teeth (LT) of the lower jaw (LJ) and the low jaw-fixed oral appliance 100b.

As described above, the molars of the upper jaw (UJ), although mounted in the upper groove 300a, are not strongly engaged onto the inside surface of the upper groove 300b unlike the molars of the lower jaw (LJ). Further, the upper-jaw teeth guide parts 311b and 312b are formed at the left and right of the upper groove 300b of the body 200b to allow the molars of the upper jaw (UJ) to escape from the upper groove 300b and be then seated back in place in the upper groove 300b. The upper-jaw teeth guide parts 311b and 312b, although formed on the outside surface of the oral appliance 100b a as shown in FIG. 12, may also be formed on the inside surface or both the outside surface and inside surface.

Wearing an oral appliance of the present invention as set forth above enables the lower jaw (LJ) to advance, allowing for smooth respiration during sleep and leading to a natural movement of the lower jaw (LJ). This may mitigate the stiffness of muscles and ligaments that may arise as the lower jaw (LJ) advances. Further, co-use of the first fastening portion by the frictional force against the molars and the second fastening portion adopting the fastening protrusion may keep the oral appliance fastened to the user's teeth although the fastening force weakens over time. As a result, a long-term use of the oral appliance of the present invention would not cause pain, malocclusion, or temporomandibular disorder, rather smoothening the air flow through the user's airway (AW) and hence enhancing the symptoms such as snoring or sleep apnea.

Although embodiments of the present invention have been described with reference to the accompanying drawings, it will be appreciated by one of ordinary skill in the art that the present disclosure may be implemented in other various specific forms without changing the essence or technical spirit of the present disclosure. Thus, it should be noted that the above-described embodiments are provided as examples and should not be interpreted as limiting. It should be noted that the scope of the present invention is defined by the appended claims rather than the described description of the embodiments and include all modifications or changes made to the claims or equivalents of the claims.

[Description of Symbols]

| | |
|---|---|
| 100: oral appliance | 100a: upper jaw-fixed oral appliance |
| 100b: low jaw-fixed oral appliance | 110, 110a, 110b: first fastening |
| | portion |

122, 122a, 122b: second fastening portion

| | |
|---|---|
| 200, 200a, 200b: body | 300, 300a, 300b: upper groove |

311b, 312b: teeth guide part
400, 400a, 400b: lower groove
411, 411b, 412b: lower jaw molar fastening groove

| | |
|---|---|
| 420b: lower jaw incisor groove | 500: guide part |

600b: tongue tip mount

What is claimed is:

1. An oral appliance, comprising:
a body having a shape corresponding to dentition;
an upper groove formed in an upper portion of the body to allow upper teeth of an upper jaw to be fitted therein;
a lower groove formed in a lower portion of the body to allow lower teeth of a lower jaw to be fitted therein and to allow the lower jaw to advance by a predetermined distance; and
a teeth guide part extending from the body to face a side surface of the upper teeth or the lower teeth, wherein the lower groove includes a first fastening portion formed to be firmly fastened to at least one of left-hand and right-hand molars of the lower teeth by friction and a second fastening portion formed to have a fastening member fastened to an interdental portion of at least one of the lower teeth.

2. The oral appliance of claim 1, wherein the body is formed of a medical material or a medical resin.

3. The oral appliance of claim 1, wherein the upper groove is formed to be spaced apart from the upper teeth not to be firmly engaged with the upper teeth.

4. The oral appliance of claim 1, wherein the lower groove further includes a lower-jaw incisor groove formed to allow the other lower teeth than the left-hand and right-hand molars to rest thereon.

5. The oral appliance of claim 4, wherein the first fastening portion and the lower-jaw incisor groove are bordered by a canine.

6. The oral appliance of claim 4, wherein the lower-jaw incisor groove is spaced apart from incisors of the lower jaw not to contact the incisors of the lower jaw.

7. The oral appliance of claim 6, wherein a distance at which the lower-jaw incisor groove is spaced apart from the incisors of the lower jaw ranges from 0.5 mm to 2.5 mm along a vertical direction.

8. The oral appliance of claim 6, wherein a distance at which the lower-jaw incisor groove is spaced apart from the incisors of the lower jaw ranges from 0.3 mm to 0.5 mm along a horizontal direction.

9. The oral appliance of claim 1, wherein the teeth guide part has a first portion extending from molars of the upper jaw or lower jaw longer and a second portion extending from incisors of the upper jaw or lower jaw, the first portion being longer than the second portion.

10. The oral appliance of claim 1, wherein the fastening member includes a wire buried in the body and a pin-shaped dental wire formed of a circular protrusion extending from the wire and attached onto an interdental portion of the lower teeth.

11. The oral appliance of claim 1, wherein the fastening member is formed of a metal, ceramic, or plastic.

12. The oral appliance of claim 1, wherein the second fastening portion is formed to overlap the first fastening portion or adjacent the first fastening portion.

13. The oral appliance of claim 1, wherein the body has an adjusting hole formed in an outside surface of where the fastening member is positioned to adjust the fastening member from an outside.

14. The oral appliance of claim 1, further comprising a tongue tip mount formed in a portion of the body to allow a user's tongue to rest thereon.

15. An oral appliance, comprising:
a body having a shape corresponding to dentition;
an upper groove formed in an upper portion of the body to allow upper teeth to be fitted therein;
a lower groove formed in a lower portion of the body to allow lower teeth of a lower jaw to be fitted therein and to allow the lower jaw to advance by a predetermined distance; and
a teeth guide part extending from the body to face a side surface of the upper teeth or the lower teeth, wherein the upper groove includes a first fastening portion formed to be firmly fastened to at least one of left-hand and right-hand molars of the upper teeth by friction and a second fastening portion formed to have a fastening member fastened to an interdental portion of at least one of the upper teeth.
